(19) United States
(12) Patent Application Publication
(10) Pub. No.: US 2025/0073374 A1
(43) Pub. Date: Mar. 6, 2025

(54) SYSTEMS AND METHODS OF ENHANCEMENTS OF THERAPEUTIC PROPERTIES OF A SOLID GRAFT MATERIAL

(71) Applicant: Royal Biologics, Hackensack, NJ (US)

(72) Inventors: Darin EWER, Providence, UT (US); Salvatore LEO, Miami, FL (US)

(73) Assignee: Royal Biologics, Hackensack, NJ (US)

(21) Appl. No.: 18/822,397

(22) Filed: Sep. 2, 2024

Related U.S. Application Data
(60) Provisional application No. 63/536,801, filed on Sep. 6, 2023.

(51) Int. Cl. A61L 27/36; A61F 2/44; A61F 2/46; A61L 27/54

(52) U.S. Cl. CPC A61L 27/3616; A61F 2/4455; A61F 2/4644; A61L 27/3687; A61L 27/3691; A61L 27/54; A61L 2300/412; A61L 2300/64; A61L 2430/02

(57) ABSTRACT

A method of enhancing therapeutic properties of a solid graft material with a fluid biologic may include obtaining the solid graft material and obtaining the fluid biologic to be fluidically infused with the solid graft material. The fluid biologic may include human platelets, activating the human platelets with an activation fluid to form activated human platelets, placing the solid graft material into a centrifuge container, adding the activated human platelets to the solid graft material in the centrifuge container, and placing the centrifuge container into a centrifuge and centrifuging the solid graft material and the activated human platelets to form an enhanced solid graft material.

### CROSS-REFERENCE TO RELATED APPLICATIONS

The present application claims the benefit of U.S. Provisional Application No. 63/536,801, filed on Sep. 6, 2023, entitled SYSTEMS AND METHODS OF ENHANCEMENTS OF THERAPEUTIC PROPERTIES OF A SOLID GRAFT MATERIAL, which is incorporated by reference as though set forth herein in its entirety.

### TECHNICAL FIELD

The present disclosure relates to solid graft materials used during human tissue grafting. More specifically, the present disclosure relates to systems and methods of enhancing therapeutic properties of a solid graft material using fluid biologically infused graft materials via centrifuging processes.

### BACKGROUND

Orthopedic health care involves conditions associated with the human musculoskeletal system. Often, orthopedic surgeons engage in surgical and non-surgical activities to treat musculoskeletal trauma, spine diseases, sports injuries, degenerative diseases, infections, tumor, and other disorders and diseases. Part of this includes creating bone grafts used to build up bone structures or fuse existing bones together. This may include multiple surgeries to initially extract portions of a patient's bone for transplant at the intended area where the graft is to take place. In other examples, bone tissue obtained from donated human cadavers may also be used. However, these measures taken to graft bone on to a patient's existing bone structure are not without risks including failures of the bone receiving the graft not healing well and/or infections from donated bone.

### SUMMARY

The various systems and methods of the present disclosure have been developed in response to the present state of the art, and in particular, in response to the problems and needs in the art that have not yet been fully solved by currently available systems and methods of enhancements of therapeutic properties of a solid graft material.

In some embodiments, a method of enhancing therapeutic properties of a solid graft material with a fluid biologic may include obtaining the solid graft material and obtaining the fluid biologic to be fluidically infused with the solid graft material. The fluid biologic may include human platelets, activating the human platelets with an activation fluid to form activated human platelets, placing the solid graft material into a centrifuge container, adding the activated human platelets to the solid graft material in the centrifuge container, and placing the centrifuge container into a centrifuge and centrifuging the solid graft material and the activated human platelets to form an enhanced solid graft material.

In the method of any preceding paragraph, the solid graft material may be one of an

autograft, an allograft, and a synthetic bone material.

In the method of any preceding paragraph, activating the human platelets with an activation additive may include adding calcium chloride (CaCl₂) to the human platelets.

In the method of any preceding paragraph, the fluid biologic may include stem cells from a patient.

In the method of any preceding paragraph, the fluid biologic may include platelet-rich plasma (PRP).

In the method of any preceding paragraph, the fluid biologic may include bone marrow aspirate from a patient.

In the method of any preceding paragraph, the method may further include using the enhanced solid graft material in a surgical procedure to facilitate fusion between a first bone portion and a second bone portion.

In some embodiments, a method of enhancing therapeutic properties of a solid graft material with a fluid biologic may include obtaining the solid graft material and obtaining the fluid biologic to be fluidically infused with the solid graft material. The fluid biologic may include human platelets, activating the human platelets with an activation fluid to form activated human platelets, and placing the solid graft material into a centrifuge container. The centrifuge container may include a body having an internal volume configured with a maximum fill level of 60 mL of the solid graft material and the fluid biologic, a threaded lid configured to threadably engage and seal the body, and a self-sealing luer port received in the threaded lid. The self-sealing luer port may be configured to engage a luer fitting of a syringe. The method may further include centrifuging the centrifuge containing during a surgical procedure to form an enhanced solid graft material.

In the method of any preceding paragraph, the centrifuge container may further include a threaded aperture configured to receive a one-way air vent.

In the method of any preceding paragraph, the fluid biologic may include platelet-rich

plasma (PRP).

In the method of any preceding paragraph, the activation fluid may be calcium chloride (CaCl₂).

In the method of any preceding paragraph, the fluid biologic may include bone marrow aspirate from a patient.

In the method of any preceding paragraph, the method may further include using the enhanced solid graft material in the surgical procedure to facilitate fusion between a first bone portion and a second bone portion.

In some embodiments, a method of enhancing therapeutic properties of a solid graft material may include obtaining, from a patient, a sample of blood. The sample of blood may contain platelets and blood-derived growth factors. The method may further include centrifuging the sample of blood to form a platelet-rich plasma, adding an activation fluid to the platelet-rich plasma to form an activated platelet-rich plasma and an activated blood-derived growth factors, and obtaining the solid graft material. During an orthopedic surgical procedure on the patient, the method may further include, adding the activated platelet-rich plasma, the activated blood-derived growth factors, and the solid graft material to a centrifuge container, centrifuging the centrifuge container to form an enhanced solid graft material, and surgically implanting the enhanced solid graft material into the patient to promote bone growth.

In the method of any preceding paragraph, the method may further include adding bone marrow aspirate to the centrifuge prior to centrifuging.

In the method of any preceding paragraph, the solid graft material may be one of an autograft, an allograft, and a synthetic bone material.

In the method of any preceding paragraph, the activation fluid may be calcium chloride (CaCl₂).

In the method of any preceding paragraph, the orthopedic surgical procedure may be a spinal fusion procedure.

In the method of any preceding paragraph, the method may further include adding stem cells from the patient to the centrifuge container prior to centrifuging.

In the method of any preceding paragraph, the centrifuge container may include a body having an internal volume to hold up to 60 mL of the solid graft material and the platelet-rich plasma, a threaded lid configured to threadably engage and seal the body, and a self-sealing luer port received in the threaded lid. The self-sealing luer port may be configured to engage a luer fitting of a syringe.

These and other features and advantages of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through the use of accompanying drawings.

### BRIEF DESCRIPTION OF THE DRAWINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the scope of the claims, the exemplary embodiments of the disclosure will be described with additional specificity and detail through use of the accompanying drawings in which:

FIG. 1 is a front view of a centrifuge container for enhancing therapeutic properties of a solid graft material according to one embodiment of the present disclosure.

FIG. 2 is a front, exploded view of a centrifuge container for enhancing therapeutic properties of a solid graft material of FIG. 1.

FIG. 3 is a side view of a body of a centrifuge container for enhancing therapeutic properties of a solid graft material according to one embodiment of the present disclosure.

FIG. 4 is a front view of the body of FIG. 3.

FIG. 5 is a cross-sectional, front view of the body of FIG. 3.

FIG. 6A is a cross-sectional, top view of the body of FIG. 3.

FIG. 6B is a cross-sectional, top view of the body of FIG. 3.

FIG. 7 is a cross-sectional, side view of the body of FIG. 3.

FIG. 8 is a perspective view of a threaded insert of a centrifuge container for enhancing therapeutic properties of a solid graft material according to one embodiment of the present disclosure.

FIG. 9 is a cross-sectional, side view of the threaded insert of FIG. 8.

FIG. 10 is a partial, cross-sectional, side view of the threaded insert of FIG. 8.

FIG. 11 is a top view of a threaded lid of a centrifuge container for enhancing therapeutic properties of a solid graft material according to one embodiment of the present disclosure.

FIG. 12 is a cross-sectional, side view of the threaded lid of FIG. 11.

FIG. 13 is a side view of a centrifuge counterbalance container for a centrifuge according to one embodiment of the present disclosure.

FIG. 14 is an exploded, side view of the centrifuge counterbalance container of FIG. 13.

FIG. 15 is a block flow diagram of a method of enhancing therapeutic properties of a solid graft material with a fluid biologic according to one embodiment of the present disclosure.

FIG. 16 is a top view of an accessory kit having a centrifuge container, a bowl, and a spatula according to an embodiment of the present disclosure.

FIG. 17 is a perspective view of a step in a method of enhancing therapeutic properties of a solid graft material with a fluid biologic according to an embodiment of the present disclosure.

FIG. 18 is a perspective view of a step in a method of enhancing therapeutic properties of a solid graft material with a fluid biologic according to an embodiment of the present disclosure.

FIG. 19A and FIG. 19B are perspective views of a step in a method of enhancing therapeutic properties of a solid graft material with a fluid biologic according to an embodiment of the present disclosure.

FIG. 20A and FIG. 20B are perspective views of a step in a method of enhancing therapeutic properties of a solid graft material with a fluid biologic according to an embodiment of the present disclosure.

FIG. 21 is a perspective view of a step in a method of enhancing therapeutic properties of a solid graft material with a fluid biologic according to an embodiment of the present disclosure.

FIG. 22A and FIG. 22B are perspective views of a step in a method of enhancing therapeutic properties of a solid graft material with a fluid biologic according to an embodiment of the present disclosure.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the disclosure will be best understood by reference to the drawings, wherein like parts are designated by like numerals throughout. It will be readily understood that the components of the disclosure, as generally described and illustrated in the FIGS. herein, could be arranged and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

The present disclosure relates to systems and methods of enhancements of therapeutic properties of a solid graft material. Those skilled in the art will recognize that the following description is merely illustrative of the principles of the technology, which may be applied in various ways to provide many alternative embodiments. To achieve the foregoing, the systems and methods of the present disclosure may provide for a graft material having enhanced therapeutic properties. In an embodiment, the solid graft material may be produced via a method that may include placing a graft material and activated human platelets from a fluid biologic into a container and placing the container into a centrifuge. The fluid biologic, in an embodiment, may be autologous, i.e.: from a current patient receiving the orthopedic health care described herein. The solid graft material may include either pieces of the patient's own bone material, synthetic bone material, and/or allograft bone material.

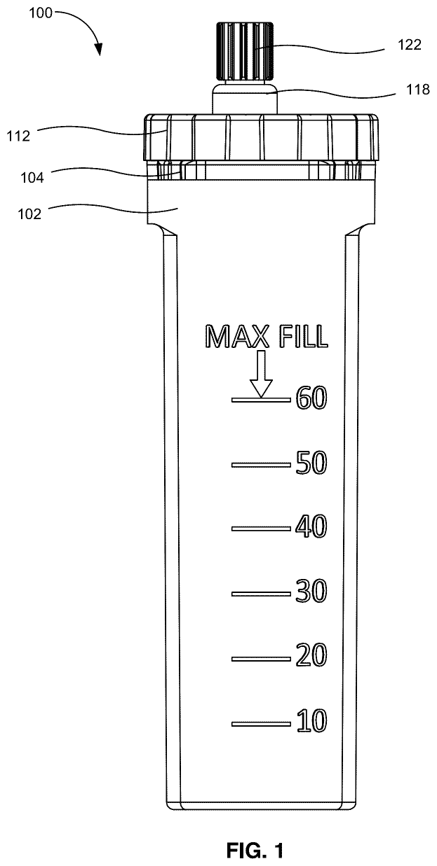

FIG. 1 is a front view of a centrifuge container 100 for enhancing therapeutic properties of a solid graft material according to one embodiment of the present disclosure. The centrifuge container 100 described herein may be used with a centrifuge to create a sold graft material that has enhanced therapeutic properties of a solid graft material.

The centrifuge container 100 may include a body 102 into which the solid graft material and a fluid biologic are placed as described herein. In an embodiment the size and shape of the body 102 may be formed to fit within an angled hole formed within a rotor of a centrifuge. In an embodiment, the volumetric capacity of the body 102 may be sufficient to form an amount of enhanced solid graft material so that a surgeon may use the enhanced sold graft material to, for example, fuse two bones to each other. In an embodiment, the body 102 may have an internal volume configured to be larger than 60 mL with a maximum fill level of 60 mL.

The centrifuge container 100 may further include a threaded insert 104 formed at an orifice of the body 102. In an embodiment, this threaded insert 104 may be press fitted into the orifice of the body 102. Alternatively, the threaded insert 104 may be affixed to the orifice of the body 102 via an adhesive. Alternatively, the threaded insert 104 may be ultrasonically welded to the orifice of the body 102. As described herein, the threaded insert 104 may include an aperture formed therethrough so that a medical professional may pass an amount of solid graft material and/or a fluid biologic into the body 102. The threaded insert 104 may include one or more threads formed onto an interior surface of the threaded insert aperture formed through the threaded insert 104.

The centrifuge container 100 may further include a threaded lid 112. The threaded lid 112 may include one or more external threads 114 that may threadably engage the threaded insert threads formed onto an interior surface of the threaded insert aperture. The threaded lid 112 may be secured to the body 102 and the threaded insert 104 after the user (e.g., a physician or other medical professional) has introduced the solid graft material into the body 102. The threaded lid 112 may also include a first threaded lid aperture 116 into which a self-sealing luer port 118 may be placed. The self-sealing luer port 118 may be either press fitted into the first threaded lid aperture 116. Alternatively, the self-sealing luer port 118 may be adhered to the first threaded lid aperture 116 by an adhesive. Alternatively, the self-sealing luer port 118 may be adhered to the first threaded aperture by ultrasonic welding. The self-sealing luer port 118 may include a luer port cap 122 that may be selectively secured to the self-sealing luer port 118 by one or more port threads 120.

The threaded lid 112 may further include a second threaded lid aperture (not shown in FIG. 1) into which a one-way air vent 126 (not shown in FIG. 1) may be placed. The one-way air vent 126 may be press fitted into the second threaded lid aperture 124. Alternatively, the one-way air vent 126 may be adhered to the second threaded lid aperture by an adhesive. Alternatively, the one-way air vent 126 may be adhered to the second threaded lid aperture by ultrasonic welding.

During operation, the user (e.g., a physician or other medical professional) may open an accessory kit containing the centrifuge container 100 and/or other medical instruments. Because of the medical nature of the centrifuge container 100 and the medical processes involved, the accessory kit 200 may include an aseptic and/or sterile barrier, thereby preventing introduction of bacteria and/or other contaminants into the systems and methods described herein. The aseptic and/or sterile barrier may ensure a desired sterility assurance level of the centrifuge container 100 and/or other medical instruments included within the accessory kit 200.

In an embodiment, the accessory kit may include the centrifuge container 100 described herein as well as, for example, a centrifuge counterbalance container 128 for use in a centrifuge to counterbalance the weight of the centrifuge container 100, a bowl 210 to hold any solid graft material, a spatula 220, a catheter, a specimen tube to receive a fluid biologic therein, a syringe, a vial comprising a fluid biologic activation fluid (e.g., calcium chloride; CaCl₂) among other medical devices used to perform the methods described herein.

The centrifuge container 100 may be open to introduce an amount of solid graft material into the body 102 of the centrifuge container 100. The graft material may be placed in a bowl 210 and poured into the body 102. In an example embodiment, the solid graft material may include relatively small pieces of autograft (e.g., human bone material from the patient), allograft (e.g., human bone material from another human besides the patient), xenograft (e.g., bone material from an animal), Magnus® by Royal Biologics®, and/or other synthetic bone material. These small pieces may be poured from the bowl 210 (e.g., the bowl 210 from the accessory kit) into the body 102 of the centrifuge container 100.

Concurrently, the health care professionals (e.g., a surgeon, a nurse, or other clinician) may obtain a sample of fluid biologic from the patient or other source. The fluid biologic may include a sample from the patient or another human. The fluid biologic may include a platelet-rich plasma (PRP) and/or bone marrow aspirate. In either case, the platelets and other blood-derived growth factors may be included within the obtained fluid biologic. These blood-derived growth factors may include, for example, insulin-like growth factor (IGF), platelet-derived growth factor (PDGF), epidermal growth factor (EGF), transforming growth factor-beta (TGF-beta), and platelet-derived epidermal growth factor (PDEGF), vascular endothelial growth factor (VEGF), and the like.

The fluid biologic may be introduced into a collection tube or other decanter that may be placed within a centrifuge in order to separate the platelets and blood-derived growth factors from the remaining portions of the blood sample. This may create a PRP as described herein. Once the separation has been completed and an activation additive (e.g., CaCl₂) may have activated the platelets, the PRP may be transferred into a syringe and/or other container for delivery into the centrifuge container 100. The additional of the activation additive may form an activated platelet-rich plasma and an activated blood-derived growth factors.

With the solid graft material sealed inside the centrifuge container 100 and with the coupling of the threaded lid 112 to the threaded insert 104, the PRP may be injected into the centrifuge container 100 through the self-sealing luer port 118. The self-sealing luer port 118 may include port threads 120 that may interface with threads formed on the syringe. An additional syringe may be used to introduce bone marrow aspirate into the centrifuge container 100. The bone marrow aspirate may be harvested from the patient or another person. The self-sealing luer port 118 may be configured to engage a luer fitting of a syringe.

The solid graft material may mix with the fluid biologic and the bone marrow aspirate (where applicable) inside the centrifuge container 100. Subsequently, the centrifuge container 100 may be centrifuged using a centrifuge machine. The centrifuge container 100 may be counterbalanced within the centrifuge using a centrifuge counterbalance container that is formed to match, generally, the weight of the centrifuge container 100. The centrifuge counterbalance container may include a centrifuge container 100 with additional weight inside its body for such a counterbalancing effect.

After being centrifuged (e.g., at around 3600 RPMs), the centrifuge container 100 may be removed to obtain the solid graft material that is now infused with the fluid biologics described herein. During operation, a surgeon may access the fluid biologic-infused solid graft material by removing the threaded lid 112 from the threaded insert 104 and pulling the infusion out from the body 102. This fluid biologic-infused solid graft material may be molded into a variety of shapes and/or certain other substrates where the fluid biologic-infused solid graft material may be accumulated such as autograft, allograft, and demineralized bone matrix. Often called a platelet-rich fibrin matrix, this may allow a surgeon to place the fluid biologic-infused solid graft material at specific location within the human anatomy in order to, for example, fuse two bones together (e.g., two vertebrae), repair a fracture, and/or fill a bone void.

The systems and methods described herein provide for simple method to enhance therapeutic properties of a solid graft material with a fluid biologic. This may be completed within a relatively short period of time immediately before or even during a surgery where bone grafts, for example, are to be made in the patient's anatomy. The systems and methods described herein may also reduce the costs associated with maintaining graft material by allowing for new graft material to be made rather than storing frozen graft material prior to surgery. Additionally, the systems and methods described herein may obviate the use any potentially toxic cryo-protectants commonly used to preserve a frozen graft. Additionally, the costs savings may be between 20 and 40 percent due to the fluid biologic-infused solid graft material not being frozen.

The centrifuge container 100 may also allow for the formation of up to 50 ccs of the fluid biologic-infused solid graft material that may be used for multiple grafts. The formation of the fluid biologic-infused solid graft material may also have enhanced healing potential especially where the fluid biologic infused into the solid graft material is the patient's own fluid biologics.

This may be due to the patient's own cells being used in some example embodiments. Still further, with the malleability of the created fluid biologic-infused solid graft material, the surgeon may have the ability to activate, create, and define graft areas for any size/shape bone void within the human body. The surgeon may also combine the fluid biologic-infused solid graft material with an allograft and/or autograft for unique bone grafting options based on any given patient's anatomy and/or clinical need discovered during surgery.

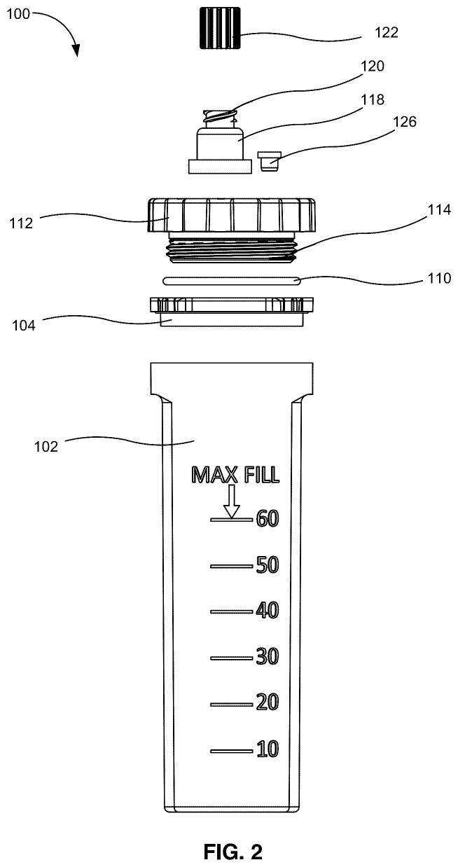

FIG. 2 is a front, exploded view of a centrifuge container 100 for enhancing therapeutic properties of a solid graft material according to an embodiment of the present disclosure. Additional elements of the centrifuge container 100, not shown in FIG. 1, may be shown in FIG. 2.

The centrifuge container 100 may include a body 102 into which the solid graft material and a fluid biologic are placed as described herein. The size and shape of the body 102 may be formed to fit within an angled hole formed within a rotor of a centrifuge. The volumetric capacity of the body 102 may be sufficient to form an amount of enhanced solid graft material sufficient to address a patient's clinical needs, for example, fuse two bones to each other.

The centrifuge container 100 may further include a threaded insert 104 formed at an aperture of the body 102. The threaded insert 104 may be press fitted into the aperture of the body 102. Alternatively, the threaded insert 104 may be affixed to the orifice of the body 102 using an adhesive. Alternatively, the threaded insert 104 may be affixed to the orifice of the body 102 using ultrasonic welding. As described herein, the threaded insert 104 may include an aperture formed therethrough so that a medical professional may pass an amount of solid graft material and/or a fluid biologic into the body 102. The threaded insert 104 may include one or more threads formed onto an interior surface of the threaded insert aperture formed through the threaded insert 104.

As shown in FIG. 2, the centrifuge container 100 may further include an o-ring 110. The o-ring 110 may be placed between the threaded insert 104 and the threaded lid 112 such that a mechanical gasket may be formed between the threaded insert 104 and threaded lid 112. With the external threads 114 of the threaded lid 112 screwed into the threaded insert threads 108 of the threaded insert 104, the o-ring 110 may seal the centrifuge container 100 from outside contaminants as well as prevent fluids such as the fluid biologic from leaking out of the centrifuge container 100 during centrifuging. The o-ring 110 may be fitted into an o-ring channel formed into the threaded insert 104.

Again, the threaded lid 112 may be secured to the body 102 and the threaded insert 104 after the solid graft material has been introduced into the body 102. The threaded insert 104 may also include a first threaded lid aperture 116 into which a self-sealing luer port 118 may be placed. The self-sealing luer port 118 may be either press fit into the first threaded lid aperture 116, adhered to the first threaded lid aperture 116 by an adhesive, or ultrasonically welded to the first threaded lid aperture 116. The self-sealing luer port 118 may include a luer port cap 122 that may be selectively secured to the self-sealing luer port 118 using one or more port threads (not shown in FIG. 1).

The threaded lid 112 may further include a second threaded lid aperture (not shown in FIG. 2) into which a one-way air vent 126 may be placed. The one-way air vent 126 may be press fitted into the second threaded lid aperture, may be adhered to the second threaded lid aperture by using an adhesive, or ultrasonically welded to the second threaded lid aperture. The one-way air vent 126 may allow air to escape the centrifuge container 100 but not enter the centrifuge container 100.

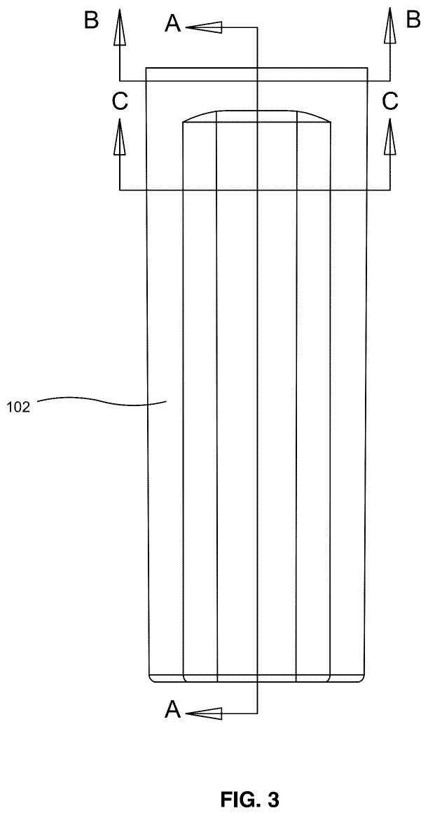

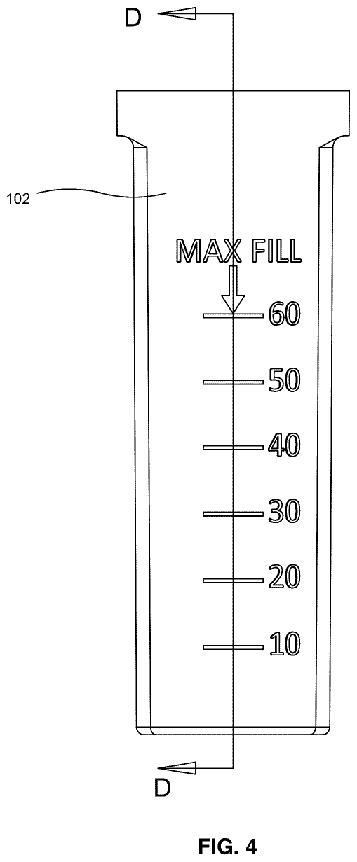

FIG. 3 is a side view of a body 102 of a centrifuge container for enhancing therapeutic properties of a solid graft material according to one embodiment of the present disclosure. FIG. 4 is a front view of the body 102. FIG. 3 and FIG. 4 include various section lines that define views referenced in other FIGS. of the present disclosure. FIG. 3 includes section line “A.” Section line “A” is a cross-sectional section line along the height of the body 102 of the centrifuge container 100 generally bifurcating the body 102 into two equal parts.

FIG. 3 also includes section line “B” which is a section line along a width of the body 102 of the centrifuge container 100 relatively close to the top of the body 102. FIG. 3 also includes section line “C” which is a section line along a width of the body 102 of the centrifuge container 100 lower than that of section line “B.” FIG. 4 includes section line “D” which is a cross-sectional section line along the height of the body 102 of the centrifuge container 100 generally bifurcating the body 102 into two equal parts. Using these section lines (A, B, C, and D), a number of other FIGS. described herein will be described.

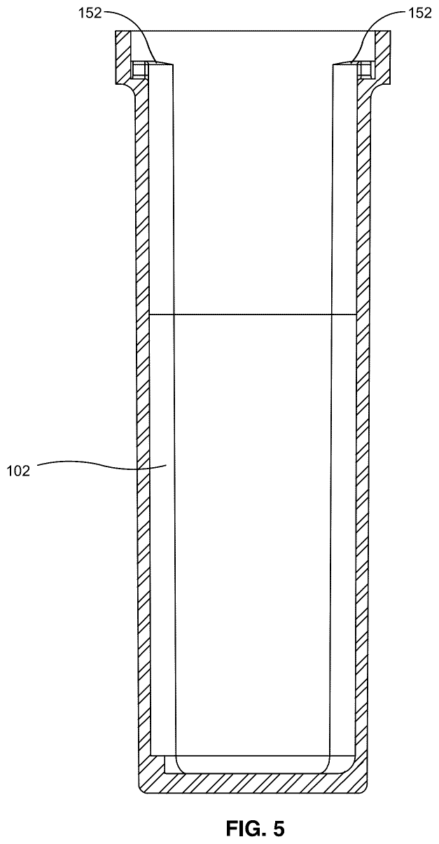

FIG. 5 is a cross-sectional, front view of the body 102. FIG. 5 includes a cross-sectional view along section line “A” of in FIG. 3. More specifically, FIG. 5 is a front view of an interior volume of the body 102. At a top portion of the body 102, a threaded insert shelf 152 may be formed. It may be appreciated that the threaded insert shelf 152 may not encompass the entire interior circumference of the body 102. The threaded insert shelf 152 may be configured to engage the threaded insert 104. At the sides of the body 102 where the threaded insert shelves 152 may be formed, the thickness of the walls of the body 102 may be thicker than other sides of the body 102. An internal diameter of the body 102 at the top of the body 102 may be approximately 1.38 inches. It is appreciated, however, that the internal diameter of the body 102 at the top of the body 102 may vary depending on the type of centrifuge used as well as the desired volumetric capacity of the centrifuge container.

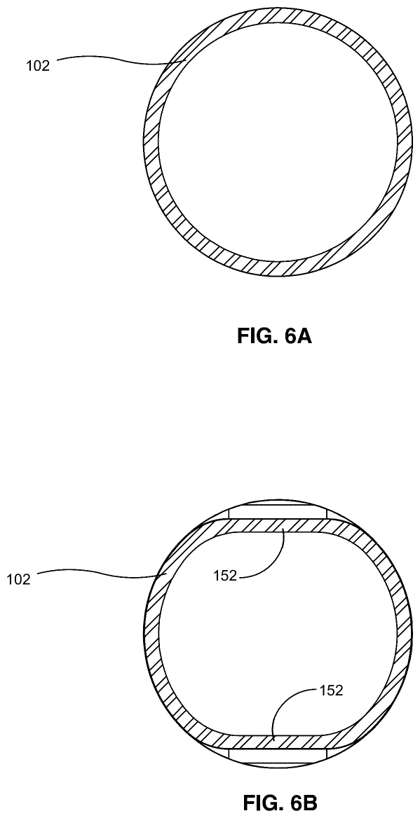

FIG. 6A is a cross-sectional, top view of the body 102. More specifically, FIG. 6A includes a cross-sectional view of the body 102 at section line “B” shown in FIG. 3. FIG. 6B is a cross-sectional, top view of the body. More specifically, FIG. 6B is a cross sectional view of the body 102 along section line “C” as shown in FIG. 3.

It may be noted that the wall thickness of FIG. 6A may be different than the wall thickness of FIG. 6B. As described herein, the threaded insert shelves 152 may not extend around the entirety of the internal circumference of the body 102. Again, the threaded insert shelves 152 are used for the threaded insert (not shown in FIGS. 6A and 6B) to rest on when inserted into the body 102 during manufacturing of the centrifuge container 100.

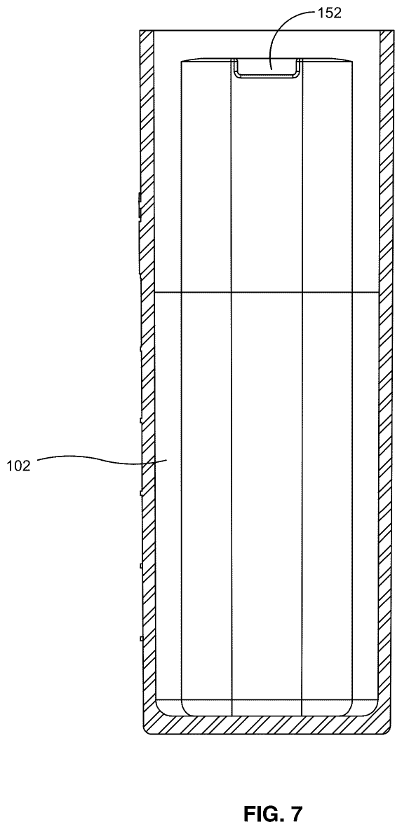

FIG. 7 is a cross-sectional, side view of the body 102. The threaded insert shelf 152 may not extend around the entire interior circumference of the body 102. However, it may be appreciated that, in some embodiments, the threaded insert shelf 152 may extend around the entire interior circumference of the body 102.

FIG. 8 is a perspective view of a threaded insert 104 of a centrifuge container 100 for enhancing therapeutic properties of a solid graft material according to one embodiment of the present disclosure. FIG. 9 is a cross-sectional, side view of the threaded insert 104. As described herein, the threaded insert 104 may include an aperture 106 that allows a user to insert the solid graft material into the body 102 of the centrifuge container 100 for preparation and centrifuging of the fluid biologic-infused solid graft material described herein.

The threaded insert 104 may include a threaded insert lip 154. The threaded insert lip 154 may rest on top of the threaded insert shelf 152. This may act as a positive stop during assembly of the centrifuge container 100 to ensure proper assembly of the threaded insert 104 relative to the body 102. Additionally, the radial orientation of the threaded insert 104 relative of the body may not matter as the threaded insert lip 154 may be formed around the entire circumference of the threaded insert 104.

As described herein, the threaded insert 104 may include threaded insert threads 108 formed on an internal diameter of the threaded insert 104. As the one or more external threads 114 of the threaded lid 112 may be screwed into the threaded insert threads 108 of the threaded insert 104, an o-ring 110 may seal the centrifuge container 100 from outside contaminants as well as prevent fluids such as the fluid biologic from leaking out of the centrifuge container 100 during centrifuging.

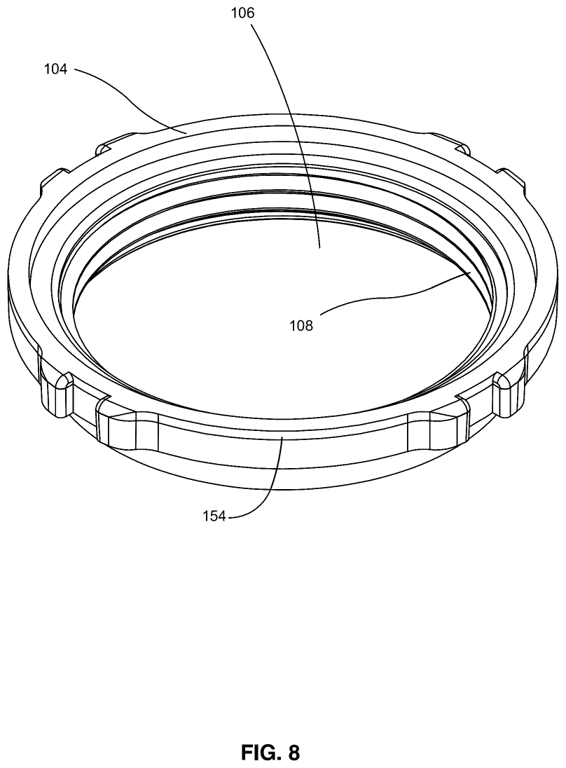

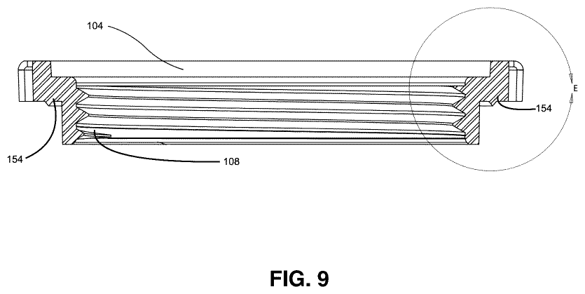

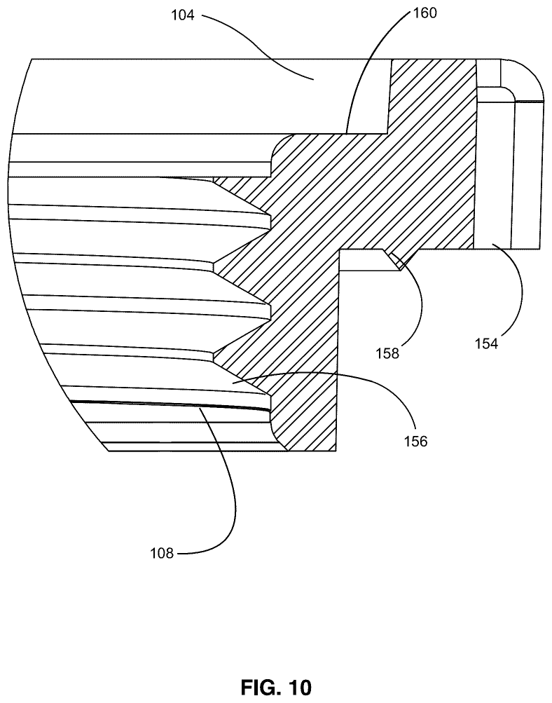

FIG. 10 is a partial, cross-sectional, side view of the threaded insert 104. More specifically, the partial, cross-sectional, side view of the threaded insert 104 of FIG. 10 is identified by detail circle “E” of FIG. 9. The threaded insert lip 154 may extend a distance away from a threaded insert external surface 156 of the threaded insert 104. The diameter of the threaded insert external surface 156 may be approximately 1.36 inches. This may allow the threaded insert external surface 156 to conform generally with an interior surface of the body 102.

A distance between the internal surface of the body 102 and the threaded insert external surface 156 may be such that a layer of adhesive may be placed between the two surfaces to secure the threaded insert 104 to the body 102. The threaded insert 104 may include a threaded insert point 158 that allows for amount of adhesive to accumulate between the top surface of a lip of the body 102 and a bottom surface of the threaded insert lip 154 so that the adhesive may secure the threaded insert 104 to the body 102 of the centrifuge container. Alternatively, the threaded insert point 158 may be configured to facilitate ultrasonic welding of the threaded insert 104 to the body 102.

FIG. 11 is a top view of a threaded lid 112 of a centrifuge container 100 for enhancing therapeutic properties of a solid graft material according to one embodiment of the present disclosure. The threaded lid 112 may include one or more external threads 114 that may engage the threaded insert threads 108 formed onto the internal surface of the threaded insert 104. The threaded lid 112 may be secured to the body 102 and threaded insert 104 after the user (e.g., a physician or other medical professional) has introduced the solid graft material into the body 102. The threaded lid 112 may also include a first threaded lid aperture 116, which may be configured to receive a self-sealing luer port 118. The self-sealing luer port 118 may be either press fitted into the first threaded lid aperture 116, adhered to the first threaded lid aperture 116 by an adhesive or ultrasonically welded to the first threaded lid aperture 116. The self-sealing luer port 118 may include a luer port cap 122 that may be selectively secured to the self-sealing luer port 118 by one or more port threads 120.

The threaded lid 112 may further include a second threaded lid aperture 124 into which a one-way air vent 126 may be received. The one-way air vent 126 may be press fitted into the second threaded lid aperture 124, the one-way air vent 126 may be adhered to the second threaded lid aperture 124 by using an adhesive, or the one-way air vent 126 may be ultrasonically welded to the second threaded lid aperture 124.

The threaded insert 104 may include an o-ring lip 160. The o-ring lip 160 may be a surface formed above the threaded insert threads 108 and along an internal diameter of the threaded insert that allows an o-ring 110 to be received within. The o-ring lip 160 may be formed to conform to a surface of the o-ring 110 such that when a threaded lid 112 is mechanically engaged with the threaded insert 104, the o-ring may prevent contaminates from entering the body 102 of the centrifuge container 100 or allow any of the solid graft material or fluid biologics to exit the centrifuge container 100.

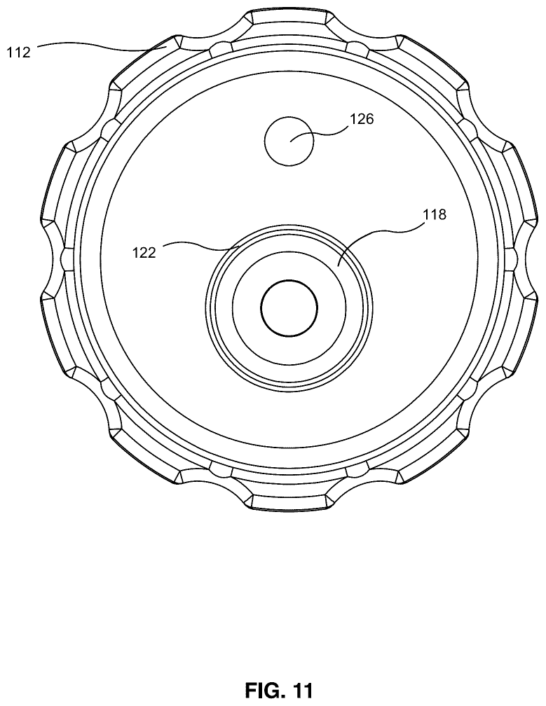

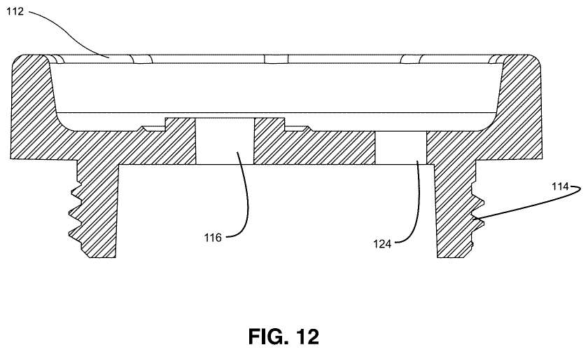

FIG. 12 is a cross-sectional, side view of the threaded lid 112. The threaded lid 112 may include a first threaded lid aperture 116 and a second threaded lid aperture 124. The self-sealing luer port 118 that may be received in the first threaded lid aperture 116 and may be secured using an adhesive, a press fit, or ultrasonic welding. The first threaded lid aperture 116 may have a diameter of approximately 0.18 inches. The second threaded lid aperture 124 may be configured to receive the one-way air vent 126. The one-way air vent 126 may be secured within the second threaded lid aperture 124 using an adhesive, a press fit, or ultrasonic welding. The second threaded lid aperture 124 may have a diameter of approximately 0.16 inches.

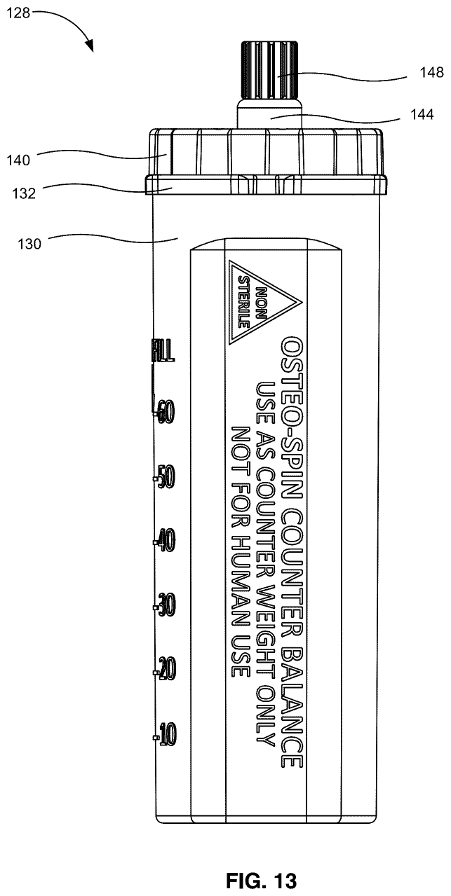

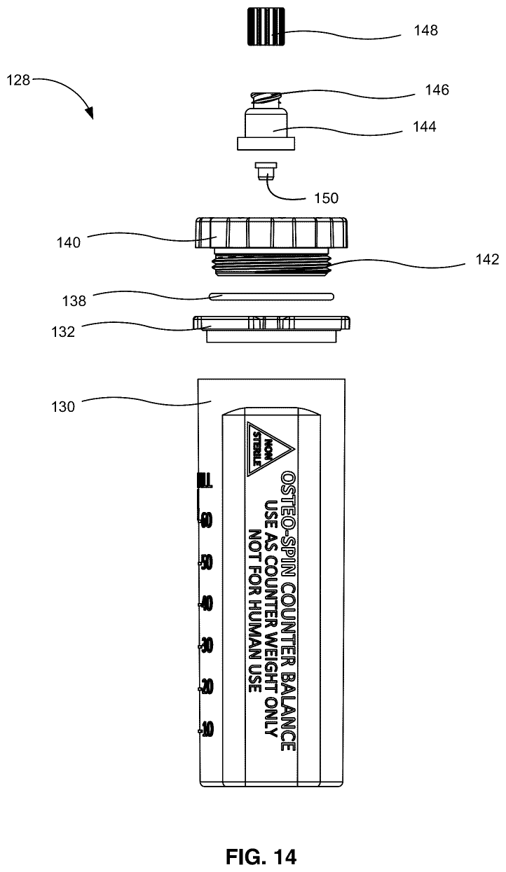

FIG. 13 is a side view of a centrifuge counterbalance container 128 for a centrifuge according to one embodiment of the present disclosure. FIG. 14 is an exploded, side view of the centrifuge counterbalance container 128. The centrifuge counterbalance container 128 may include similar elements and devices as described in connection with the centrifuge container 100 described herein. Indeed, in order to provide a counterbalance within the centrifuge when the centrifuge container 100 is being centrifuged, the centrifuge counterbalance container 128 may have similar elements in order to provide a correct amount of weight.

For example, the centrifuge counterbalance container 128 may include a centrifuge body 130 into which, for example, an amount of weight may be placed that is generally equal to a weight of the solid graft material and a fluid biologics within the centrifuge container 100. The size and shape of the centrifuge body 130 may be formed to fit within an angled hole formed within a rotor of a centrifuge opposite the angled hole configured to receive the centrifuge container 100. The volumetric capacity of the centrifuge body 130 may be sufficient to hold the necessary weight (either as a fluid or a solid) used to counterbalance the centrifuge container 100 with the centrifuge counterbalance container 128. The volumetric capacity of the centrifuge body 130 may be larger than 60 mL with a maximum suggested fill level to be at 60 mL.

The centrifuge counterbalance container 128 may further include a counterbalance threaded insert 132 formed at an orifice of the centrifuge body 130. The counterbalance threaded insert 132 may be press fitted into the orifice of the centrifuge body 130. Alternatively, the counterbalance threaded insert 132 may be affixed to the orifice of the centrifuge body 130 using an adhesive. Alternatively, the counterbalance threaded insert 132 may be ultrasonically welded to the orifice of the centrifuge body 130.

The counterbalance threaded insert 132 may include a counter balance threaded insert aperture formed therethrough so that a medical professional may pass an amount of solid material and/or fluid material into the centrifuge body 130 so as to act as a counterweight to the solid graft material and fluid biologics placed in the centrifuge container 100. The counterbalance threaded insert 132 may include one or more threads formed onto an interior surface of the counter balance threaded insert aperture formed through the counterbalance threaded insert 132. The counterbalance threaded insert 132 may also include a threaded insert shelf, a threaded insert lip, a threaded insert external surface, and a threaded insert point similar to that described in connection with the threaded insert 104. The counterbalance threaded insert 132 may also be fitted with a counterbalance o-ring 138 that may serve similar functions as that of the o-ring 110 of the centrifuge container 100.

The centrifuge counterbalance container 128 may further include a counterbalance threaded lid 140. The counterbalance threaded lid 140 may include one or more counterbalance external threads 142 that may engage with the counterbalance threaded insert threads formed onto the internal surface of the counterbalance threaded insert 132. The counterbalance threaded lid 140 may be secured to the centrifuge body 130 and counterbalance threaded insert 132 after the user (e.g., a physician or other medical professional) has introduced the solid and/or fluidic material into the centrifuge body 130 to act as a counterweight.

The counterbalance threaded lid 140 may also include a first threaded lid aperture 116 into which a counterbalance self-sealing luer port 144 may be placed. The counterbalance self-sealing luer port 144 may be press fitted into the first threaded lid aperture 116, adhered to the first threaded lid aperture 116 by an adhesive, or ultrasonically welded to the first threaded lid aperture 116. The counterbalance self-sealing luer port 144 may include a counterbalance luer port cap 148 that may be selectively secured to the counterbalance self-sealing luer port 144 via one or more counterbalance port threads 146.

The counterbalance threaded lid 140 may further include a second threaded lid aperture 124 into which a counterbalance one-way air vent 150 may be received. The counterbalance one-way air vent 150 may be press fitted into the second threaded lid aperture 124, adhered to the second threaded lid aperture 124 by an adhesive, or ultrasonically welded to the second threaded lid aperture 124.

During operation and after the surgeon has completed the processes of placing the solid graft material and fluid biologic into the centrifuge container 100, the centrifuge counterbalance container 128 may be prepared by adding an amount of weight into the centrifuge counterbalance container 128 that would, generally, counterbalance the wight of the centrifuge container 100.

As described herein, the centrifuge container 100 and centrifuge counterbalance container 128 may be placed within the centrifuge opposite each other so that they may counterbalance each other. Because the centrifuge container 100 and its contents may be rotated within the centrifuge at or near 3600 RPMs, the balancing of weight between the centrifuge counterbalance container 128 and centrifuge container 100 may necessarily be within a certain amount so that the centrifuge may operate correctly during the centrifuging process.

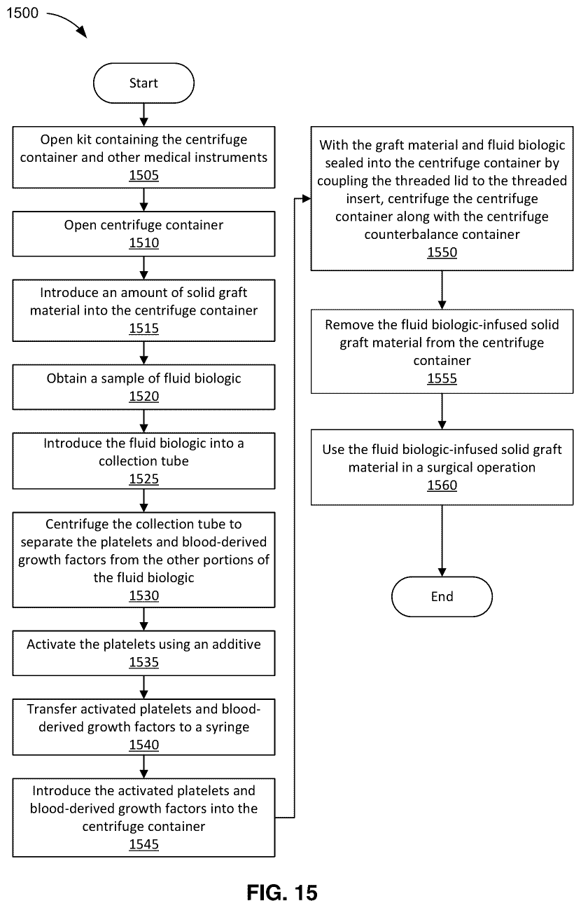

FIG. 15 is a block flow diagram of a method of enhancing therapeutic properties of a solid graft material with a fluid biologic according to one embodiment of the present disclosure. The method 1500 may describe how a surgeon or other medical practitioner may prepare a fluid biologic-infused solid graft material used to form a graft within a patient during surgery using the centrifuge container described herein.

The method 1500 may include, at block 1505, opening an accessory kit 200 into which the centrifuge container and other medical instruments may have been placed. Because of the medical nature of the centrifuge container and the medical processes involved, the centrifuge container and these medical instruments may have been previously sanitized and/or sterilized thereby preventing the introduction of bacteria or other contaminants into the systems and methods described herein. The accessory kit 200 may include the centrifuge container 100 described herein as well as, for example, a centrifuge counterbalance container 128 for use in a centrifuge to counterbalance the weight of the centrifuge container 100, a bowl 210 to hold any solid graft material, a spatula 220, a catheter, a specimen tube to receive a fluid biologic therein, a syringe, a vial comprising a fluid biologic activation fluid (e.g., calcium chloride; CaCl₂) among other medical devices used to perform the methods described herein.

At block 1510, the method 1500 may include opening the centrifuge container 100 and, at block 1515, introducing an amount of solid graft material into the body 102 of the centrifuge container 100. In an example embodiment, the graft material may be placed in a bowl 210 that may be included within the accessory kit 200 and poured into the body 102 of the centrifuge container 100. In an example embodiment, the solid graft material may include relatively small pieces of autograft (e.g., human bone material from the patient), allograft (e.g., human bone material from another human besides the patient), xenograft (e.g., bone material from an animal), Magnus® by Royal Biologics®, and/or synthetic bone material. These small pieces may be measured and then poured from the bowl 210 into the body 102 of the centrifuge container 100.

At block 1520 of the method 1500, the health care professionals (e.g., a surgeon, a nurse or other clinician) may obtain a sample of fluid biologic from the patient or other source. The fluid biologic may include a sample from the patient or another human. In an embodiment, the fluid biologic may include a platelet-rich plasma (PRP), stem cells, and/or bone marrow aspirate. Platelets and other blood-derived growth factors may be included within the obtained fluid biologic. These blood-derived growth factors may include, for example, insulin-like growth factor (IGF), platelet-derived growth factor (PDGF), epidermal growth factor (EGF), transforming growth factor-beta (TGF-beta), and platelet-derived epidermal growth factor (PDEGF), vascular endothelial growth factor (VEGF), and the like.

At block 1525, the method 1500 may include introducing the fluid biologic into a collection tube or other decanter that may be placed within a centrifuge in order to separate the platelets and blood-derived growth factors from the remaining portions of the blood sample. By centrifuging the fluid biologic at block 1530, the PRP may be created as described herein. Once the separation may be completed, an activation additive (e.g., CaCl₂) may be used to activate the platelets at block 1535. At block 1540, the PRP and blood-derived growth factors may be transferred into a syringe. The syringe may then be used to introduce the activated platelets and blood-derived growth factors into the centrifuge container at block 1545. This may be achieved by interfacing a tip of the syringe with the self-sealing luer port that includes the port threads used to interface with threads at the tip of the syringe.

Once the fluid biologics have been injected into the centrifuge container, the solid graft material mixed with the fluid biologic, stem cells, and/or bone marrow aspirate may be sealed inside the centrifuge container. The centrifuge container may be centrifuged using a centrifuge machine at block 1550 to form an enhanced solid graft material. The enhanced solid graft material may include osteogenic cells, osteoinductive signals, and/or osteoconductive scaffolds.

In an embodiment, the centrifuge container may be counterbalanced within the centrifuge using a centrifuge counterbalance container that may be formed to match, generally, the weight of the centrifuge container. In an embodiment, the centrifuge counterbalance container may include a centrifuge container with additional weight inside its body for such a counterbalancing effect.

At block 1555, the method 1500 may include removing the enhanced solid graft material from the centrifuge container. A surgeon may do this prior to or while in an operating theater in an example embodiment. At block 1560, the enhanced solid graft material may be used in a surgical procedure. More specifically, the surgical procedure may be an orthopedic surgical procedure. More specifically, the surgical procedure may be a spinal fusion procedure. This may include accessing a bone fusion site and placing the fluid biologic-infused solid graft material at a location where two bones (e.g., vertebrae), two bone portions (e.g., due to a fracture), and/or bone voids are to be fused together and/or filled. The enhanced solid graft material may be surgically implanted into a patient to promote bone growth. At this point, the method 1500 may end with the surgeon throwing the centrifuge container away as well as any other biological hazardous materials used during the method.

A method for preparation of Platelet-Rich Fibrin Membrane (PRFM) and Platelet-Rich Fibrin Gel (PRFG) from a small sample of blood at a patient point of care may include the following steps:
1. Separate and Concentrate the Platelets: Draw a patient's blood into one or more blood collection tubes and spin in a centrifuge for approximately six minutes. After centrifugation, concentrated platelets and plasma may be separated from other blood components to form platelet-rich-plasma (PRP). PRP may then be added to activation fluid (e.g., calcium chloride; CaCl₂), thereby activating the PRP and the growth factors.
2. Produce the Platelet-Rich Fibrin Membrane (PRFM) Graft: Resuspend the concentrated platelets into the plasma; then transfer sufficient volume into a membrane vial and spin in the centrifuge for approximately twenty-five minutes. After centrifugation, the membranes may be ready to be delivered to a sterile field.
3. Mix Platelet-Rich Fibrin Gel (PRFG) with Bone Grafting Material: Using a remaining tube from the centrifuge spin, mix the concentrated platelets and plasma with bone grafting material manually or via centrifugation for desired handling characteristics.

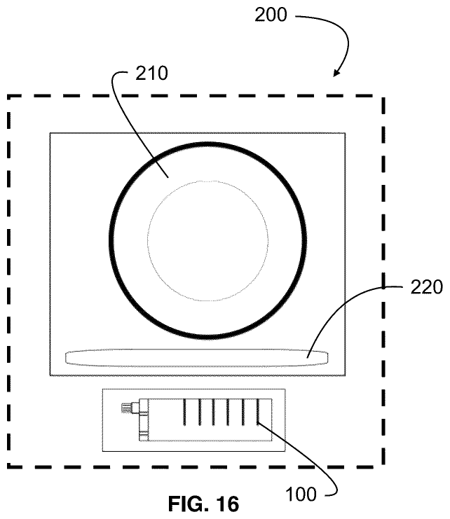

FIG. 16 is a top view of an accessory kit 200 having a centrifuge container 100, a bowl 210, and a spatula 220 according to an embodiment of the present disclosure. In other embodiments, the accessory kit 200 may also include a centrifuge counterbalance container 128, a catheter, a specimen tube to receive a fluid biologic therein, a syringe, a vial comprising a fluid biologic activation fluid (e.g., calcium chloride; CaCl₂) among other medical devices used to perform the methods described herein. The accessory kit 200 may include an aseptic and/or sterile barrier, thereby preventing introduction of bacteria and/or other contaminants into the systems and methods described herein. The aseptic and/or sterile barrier may ensure a desired sterility assurance level of the centrifuge container 100 and/or other medical instruments included within the accessory kit 200. The accessory kit 200 and its contents, including, but not limited to, the centrifuge container 100, bowl 210, and spatula 220, may be configured as one-time use disposable devices.

A method of enhancing therapeutic properties of a solid graft material with a fluid biologic may be configured to be used for safe and rapid preparation of autologous platelet-rich-plasma (PRP) from a small sample of blood at the patient point of care. The PRP may be mixed with autograft and/or allograft bone prior to application to a bony defect for improving handling characteristics. The method may include the following steps:

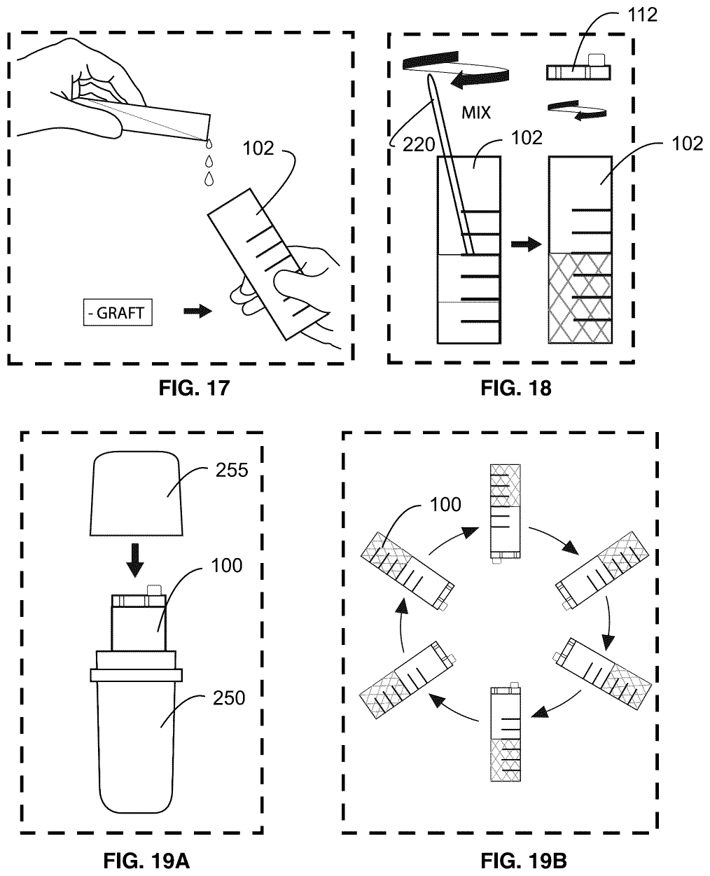

FIG. 17 is a perspective view of a step in a method of enhancing therapeutic properties of a solid graft material with a fluid biologic according to an embodiment of the present disclosure. Prepare PRFG as described herein. Remove the centrifuge container 100 from the accessory kit 200. Remove the threaded lid 112 and pour the PRFG into the body 102 of the centrifuge container 100. Alternatively, add the PRP via sterile syringe fitted with a sterile blunt tip needle through the self-sealing luer port 118 into the body 102 of the centrifuge container 100. Add desired allograft, autograft, and/or other bone grafting material to the body 102. Add bone marrow aspirate to the body 102, if desired.

FIG. 18 is a perspective view of a step in a method of enhancing therapeutic properties of a solid graft material with a fluid biologic according to an embodiment of the present disclosure. Remove the spatula 220 and manually mix the graft with the PRFG. Secure the threaded lid 112 to the body 102 and remove the luer port cap 122.

FIG. 19A and FIG. 19B are perspective views of a step in a method of enhancing therapeutic properties of a solid graft material with a fluid biologic according to an embodiment of the present disclosure. Place the centrifuge container 100 into centrifuge buckets 250 of the centrifuge ensuring that the centrifuge container is equally balanced in opposite compartments by using appropriately weighted centrifuge counterbalance containers 128. Place the centrifuge bucket cap 255 securely onto the centrifuge bucket 250. Centrifuge the centrifuge containers 100 at approximately 3600 RPMs for approximately 25 minutes.

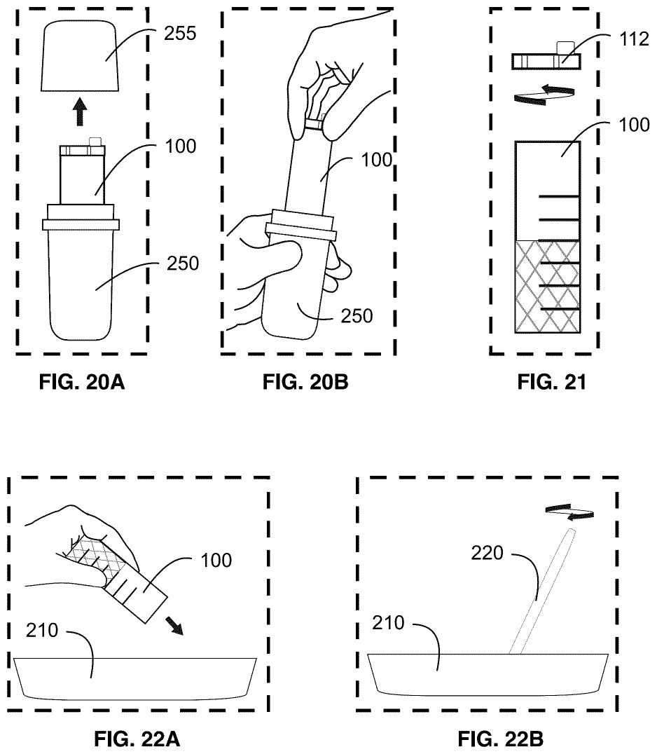

FIG. 20A and FIG. 20B are perspective views of a step in a method of enhancing therapeutic properties of a solid graft material with a fluid biologic according to an embodiment of the present disclosure. After centrifugation is complete, the centrifuge bucket cap 255 may be removed from the centrifuge bucket 250 and the centrifuge container 100 may be removed from the centrifuge bucket 250.

FIG. 21 is a perspective view of a step in a method of enhancing therapeutic properties of a solid graft material with a fluid biologic according to an embodiment of the present disclosure. The threaded lid 112 may be removed from the body 102.

FIG. 22A and FIG. 22B are perspective views of a step in a method of enhancing therapeutic properties of a solid graft material with a fluid biologic according to an embodiment of the present disclosure. The PRFM graft may be poured out of the body 102, into the bowl 210. If necessary, the spatula 220 may be used to aid in the removal of the graft from the body 102. The spatula 220 may be used to the graft to a desired consistency in the bowl 210.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, FIG., or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the disclosure.

While specific embodiments and applications of the present disclosure have been illustrated and described, it is to be understood that the disclosure is not limited to the precise configuration and components disclosed herein. Various modifications, changes, and variations which will be apparent to those skilled in the art may be made in the arrangement, operation, and details of the methods and systems of the present disclosure set forth herein without departing from its spirit and scope.

1. A method of enhancing therapeutic properties of a solid graft material with a fluid biologic, the method comprising:
obtaining the solid graft material;
obtaining the fluid biologic to be fluidically infused with the solid graft material, the fluid biologic comprising human platelets;
activating the human platelets with an activation fluid to form activated human platelets;
placing the solid graft material into a centrifuge container;
adding the activated human platelets to the solid graft material in the centrifuge container; and
placing the centrifuge container into a centrifuge and centrifuging the solid graft material and the activated human platelets to form an enhanced solid graft material.

2. The method of claim 1, wherein the solid graft material is one of an autograft, an allograft and a synthetic bone material.

3. The method of claim 1, wherein activating the human platelets with an activation additive includes adding calcium chloride (CaCl₂) to the human platelets.

4. The method of claim 1, wherein the fluid biologic includes stem cells from a patient.

5. The method of claim 1, wherein the fluid biologic includes platelet-rich plasma (PRP).

6. The method of claim 1, wherein the fluid biologic includes bone marrow aspirate from a patient.

7. The method of claim 1, further comprising using the enhanced solid graft material in a surgical procedure to facilitate fusion between a first bone portion and a second bone portion.

8. A method of enhancing therapeutic properties of a solid graft material with a fluid biologic, the method comprising:
obtaining the solid graft material;
obtaining the fluid biologic to be fluidically infused with the solid graft material, the fluid biologic comprising human platelets;
activating the human platelets with an activation fluid to form activated human platelets;
placing the solid graft material into a centrifuge container wherein:
the centrifuge container comprises:
a body having an internal volume configured with a maximum fill level of 60 mL of the solid graft material and the fluid biologic;
a threaded lid configured to threadably engage and seal the body; and
a self-sealing luer port received in the threaded lid, the self-sealing luer port configured to engage a luer fitting of a syringe; and
centrifuging the centrifuge container during a surgical procedure to form an enhanced solid graft material.

9. The method of claim 8, wherein the centrifuge container further comprises a threaded lid aperture configured to receive a one-way air vent.

10. The method of claim 8, wherein the fluid biologic includes platelet-rich plasma (PRP).

11. The method of claim 8, wherein the activation fluid is calcium chloride (CaCl₂).

12. The method of claim 8, wherein the fluid biologic includes bone marrow aspirate from a patient.

13. The method of claim 8, further comprising using the enhanced solid graft material in the surgical procedure to facilitate fusion between a first bone portion and a second bone portion.

14. A method of enhancing therapeutic properties of a solid graft material, the method comprising:
obtaining from a patient a sample of blood, wherein the sample of blood contains platelets and blood-derived growth factors;
centrifuging the sample of blood to form a platelet-rich plasma;
adding an activation fluid to the platelet-rich plasma to form an activated platelet-rich plasma and activated blood-derived growth factors;
obtaining the solid graft material; and
during an orthopedic surgical procedure on the patient:
adding the activated platelet-rich plasma, the activated blood-derived growth factors, and the solid graft material to a centrifuge container;
centrifuging the centrifuge container to form an enhanced solid graft material; and
surgically implanting the enhanced solid graft material into the patient to promote bone growth.

15. The method of claim 14, further comprising adding bone marrow aspirate to the centrifuge container prior to centrifuging.

16. The method of claim 14, wherein the solid graft material is one of an autograft, an allograft and a synthetic bone material.

17. The method of claim 14, wherein the activation fluid is calcium chloride (CaCl₂).

18. The method of claim 14, wherein the orthopedic surgical procedure is a spinal fusion procedure.

19. The method of claim 14, further comprising adding stem cells from the patient to the centrifuge container prior to centrifuging.

20. The method of claim 14, wherein the centrifuge container comprises:
a body having an internal volume to hold up to 60 mL of the solid graft material and the platelet-rich plasma;
a threaded lid configured to threadably engage and seal the body; and
a self-sealing luer port received in the threaded lid, the self-sealing luer port configured to engage a luer fitting of a syringe.
